(19) United States
(12) Patent Application Publication
(10) Pub. No.: US 2017/0296143 A1
(43) Pub. Date: Oct. 19, 2017

(54) ROTARY LINEAR PROBE

(71) Applicant: GE ULTRASOUND KOREA LTD., Seongnam-si (KR)

(72) Inventors: Jeong Seok KIM, Seongnam-si, Gyeonggi-do (KR); Jong Gun LEE, Seongnam-si, Gyeonggi-do (KR); Yong Ho HYUN, Seongnam-si, Gyeonggi-do (KR)

(21) Appl. No.: 15/490,326

(22) Filed: Apr. 18, 2017

Related U.S. Application Data
(63) Continuation of application No. PCT/KR2016/006239, filed on Jun. 13, 2016.

(30) Foreign Application Priority Data
Apr. 18, 2016 (KR) 10-2016-0046816

(51) Int. Cl. A61B 8/00; A61B 8/08; G01S 15/89; G01S 7/521

(52) U.S. Cl. CPC A61B 8/4461; G01S 15/894; A61B 8/4477; A61B 8/461; A61B 8/54; A61B 8/483; G01S 7/521

(57) ABSTRACT

The present invention relates to a rotary linear probe. The rotary linear probe includes a rotatable element in the form of a linear rod along a rotation axis and a plurality of ultrasonic modules positioned in a row along the rotation axis on the rotatable element. The use of the rotary linear probe can provide better diagnostic image information based on image signals with improved 2D/3D circular image quality.

### TECHNICAL FIELD

The present invention relates to a rotary linear probe, and more specifically to a probe and an ultrasonic diagnostic system, each of which uses a plurality of rotatable linear ultrasonic modules to acquire an image of a region of interest during rotation.

### BACKGROUND ART

Generally, an ultrasonic probe has a conversion element consisting of a large population of ultrasonic oscillators. The ultrasonic probe emits ultrasonic waves to an object, receives signals reflected from the object, and converts the reflected signals into electric signals. An ultrasonic diagnostic system including the ultrasonic probe is particularly suitable for medical applications, such as detection of foreign matter in organisms, determination of the degree of lesions, observation of tumors, and prenatal observation. For more accurate medical judgement, techniques are currently being developed to obtain three-dimensional images by turning conversion elements for ultrasonic diagnosis.

### DETAILED DESCRIPTION OF THE INVENTION

### Technical Problem

It is a first object of the present invention to provide a rotary linear probe using a plurality of rotatable linear ultrasonic modules to acquire an image of a region of interest during rotation.

It is a second object of the present invention to provide an ultrasonic diagnostic system using a plurality of rotatable linear ultrasonic modules to acquire an image of a region of interest during rotation.

### Technical Solution

One aspect of the present invention provides a rotary linear probe including a rotatable element in the form of a linear rod along a rotation axis and a plurality of ultrasonic modules positioned in a row along the rotation axis on the rotatable element.

According to an embodiment of the present invention, the plurality of ultrasonic modules are capable of independent focusing.

According to an embodiment of the present invention, the plurality of ultrasonic modules may focus in pairs on different depths corresponding to predetermined planes perpendicular to the rotation axis during rotation to acquire two-dimensional (2D) circular cross-sectional image data.

According to an embodiment of the present invention, the plurality of ultrasonic modules may focus on the same depth in a direction perpendicular to the rotation axis during rotating to acquire cylindrical three-dimensional (3D) circular image data.

According to an embodiment of the present invention, each of the ultrasonic modules may include a transducer adapted to transmit and receive ultrasonic waves and a control unit adapted to adjust the transmission angle of the ultrasonic waves from the transducer.

According to an embodiment of the present invention, the rotary linear probe may further include a rotary motor adapted to rotate the rotatable element.

A further aspect of the present invention provides an ultrasonic diagnostic system including a rotatable element in the form of a linear rod along a rotation axis, a plurality of ultrasonic modules positioned in a row along the rotation axis on the rotatable element, and an image processing unit adapted to create an ultrasound image based on image data acquired during rotation of the ultrasonic modules.

According to an embodiment of the present invention, the ultrasonic diagnostic system may further include a display unit adapted to display the ultrasound image.

### Effects of the Invention

According to the present invention, the use of the rotary linear probe can provide better diagnostic image information based on image signals with improved 2D/3D circular image quality.

### BRIEF DESCRIPTION OF THE DRAWINGS

FIG. 1 illustrates a rotary linear probe according to one embodiment of the present invention,

FIG. 2 shows a process for creating an image by using a rotary linear probe according to one embodiment of the present invention,

FIG. 3 shows a process for creating a 2D image by using a rotary linear probe according to one embodiment of the present invention,

FIG. 4 shows a process for creating a 3D image by using a rotary linear probe according to one embodiment of the present invention,

FIG. 5 is a block diagram of an ultrasonic diagnostic system according to one embodiment of the present invention, and

FIG. 6 is a diagram showing an ultrasonic diagnostic system according to one embodiment of the present invention.

### BEST MODE FOR CARRYING OUT THE INVENTION

A rotary linear probe according to one embodiment of the present invention includes a rotatable element in the form of a linear rod along a rotation axis and a plurality of ultrasonic modules positioned in a row along the rotation axis on the rotatable element.

### Mode for Carrying Out the Invention

Prior to the detailed description of the present invention, an overview of the solutions proposed by the present invention or the core of the technical spirit of the present invention will be presented for convenience of understanding.

A rotary linear probe according to one embodiment of the present invention includes a rotatable element in the form of a linear rod along a rotation axis and a plurality of ultrasonic modules positioned in a row along the rotation axis on the rotatable element.

Embodiments of the present invention will now be described in detail with reference to the accompanying drawings such that those skilled in the art can readily practice the invention. However, it will be obvious to those skilled in the art in the art that these embodiments are provided to more specifically explain the invention and are not intended to limit the scope of the invention.

The constitutions of the present invention for clarifying the solutions proposed by the present invention will be described in detail based on preferred embodiments with reference to the accompanying drawings. In the drawings, the same elements are denoted by the same reference numerals even though they are depicted in different drawings. Thus, elements in other drawings can be referred to as being necessary for a description of the other drawings. In the detailed description of the principle of operation of preferred embodiments of the present invention, detailed explanations of related known functions or constructions are omitted when it is deemed that they may unnecessarily obscure the essence of the present invention.

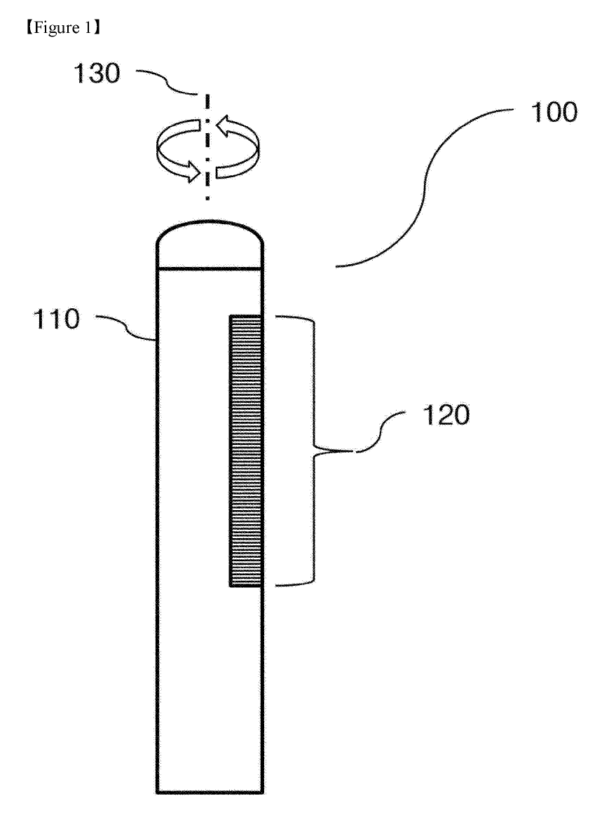

FIG. 1 illustrates a rotary linear probe 100 according to one embodiment of the present invention.

The rotary linear probe 100 includes a rotatable element 110 and a plurality of ultrasonic modules 120. The rotary linear probe 100 may further include a rotary motor.

The rotatable element 110 is in the form of a linear rod along a rotation axis 130.

More specifically, the probe in the form of a linear rod is insertable into the human body to diagnose diseases, such as prostate, coloanal, and cervical diseases. The rotatable element 110 is advantageous in acquiring an ultrasound image of its surrounding. The rotatable element can rotate a full 360 degrees around the rotation axis. This enables acquisition of a circular image.

The rotary linear probe may further include a rotary motor adapted to rotate the rotatable element 110. The rotary motor can be controlled such that the rotatable element rotates a full 360 degrees.

The plurality of ultrasonic modules are positioned in a row along the rotation axis 130 on the rotatable element.

More specifically, the plurality of ultrasonic modules 120 are used to acquire image data with improved image quality and are positioned in a row along the rotation axis. The plurality of ultrasonic modules 120 are capable of independent focusing. Unlike the prior art, the plurality of ultrasonic modules 120 are not single devices but linear multi-active devices.

Each of the ultrasonic modules 120 may include a transducer adapted to transmit and receive ultrasonic waves and a control unit adapted to adjust the transmission angle of the ultrasonic waves from the transducer. The transducer transmits ultrasonic waves to a particular location and receives ultrasonic signals reflected from the location to create an ultrasound image of the location. The control unit adjusts the transmission angle of the ultrasonic waves from the transducer to acquire 2D or 3D image data.

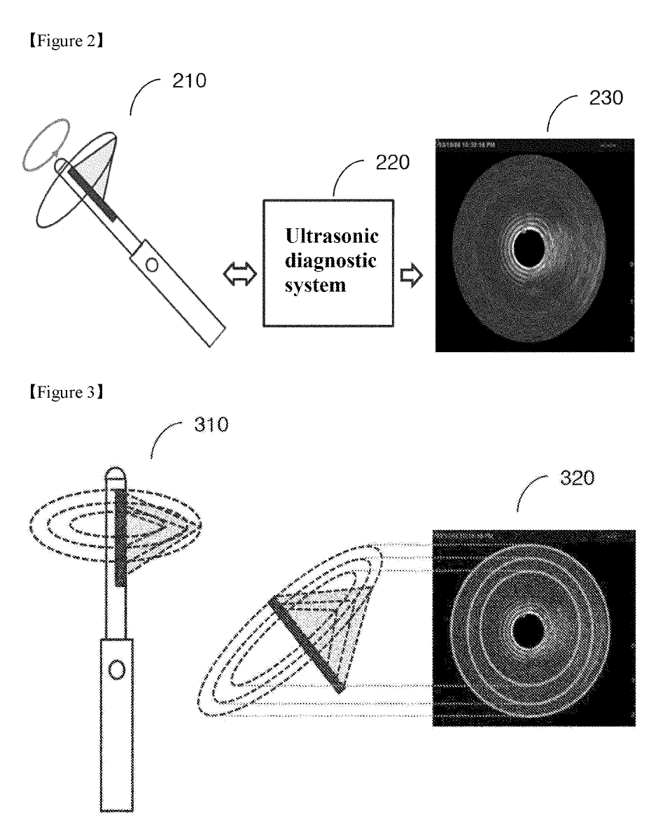

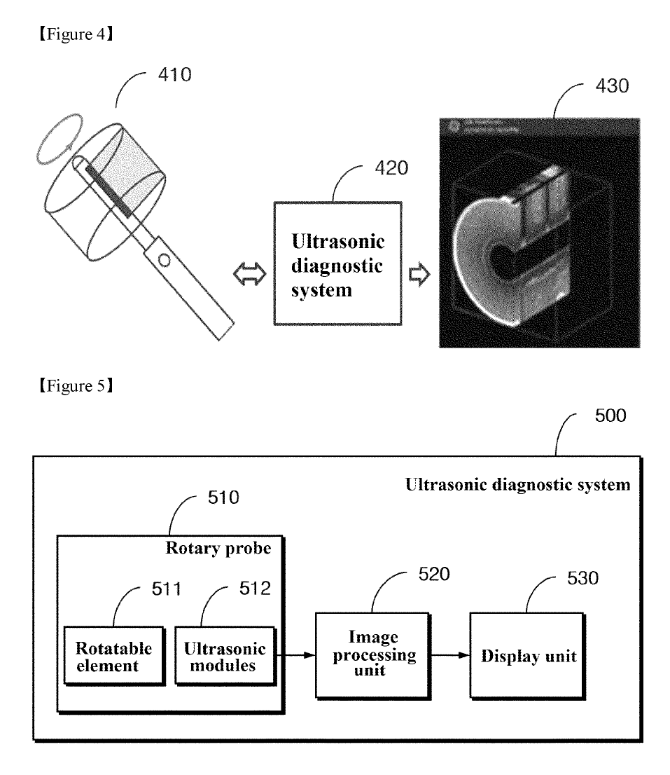

A specific process for acquiring 2D or 3D circular image data by using the rotatable and independently focusable rotary linear probe will be explained with reference to FIGS. 2 to 4.

As shown in FIG. 2, the rotary linear probe focuses on a particular location at a particular depth during rotation 210 to obtain a circular scan line. Based on the cumulation of such data, echo signal data 220 obtained by scanning the circular region are converted into a 2D circular image 230.

The use of the plurality of ultrasonic modules enables rapid acquisition of image data on one circular cross-section, as shown in FIG. 3. To this end, the plurality of ultrasonic modules may focus in pairs on different depths corresponding to predetermined planes perpendicular to the rotation axis during rotation 310 to acquire 2D circular cross-sectional image data 320. Two or more of the ultrasonic modules can focus on the same location. The resolution of the image may be increased by increasing the number of the focusing ultrasonic modules.

The depth region to be observed is determined by transmission and reception focusing. That is, depending on the particular depth, transmission and reception focusing is effected to construct a 2D circular image. Particularly, the quality of the image can be significantly improved by dynamic reception focusing compared to the quality of circular images obtained using conventional single-element devices.

The ultrasonic modules focusing on different depths pair symmetrically about the center. That is, two ultrasonic modules disposed at the most distant positions from the center pair with each other and two ultrasonic modules positioned closest to the center pair with each other. This pairing can reduce the interference between the ultrasonic modules and enables rapid acquisition of 2D circular cross-sectional image data with improved quality on a region forming a predetermined plane perpendicular to the rotation axis.

This concept can be extended to the acquisition of a 3D circular image. 2D images can be continuously obtained by adjusting the transmission angle of the ultrasonic waves from the ultrasonic modules such that the ultrasonic modules focus on different locations. A 3D circular image can be acquired by cumulatively adding up the 2D images.

The plurality of ultrasonic modules 120 may focus on the same depth in a direction perpendicular to the rotation axis during rotation 410 to acquire cylindrical 3D circular image data. In this case, the ultrasonic modules are required to transmit ultrasonic waves at the same angle.

The independently controllable ultrasonic modules can be utilized in various forms, if needed. The quality of an ultrasound image of a particular region can be increased by increasing the number of the ultrasonic modules focusing on the corresponding region than on other regions. That is, a larger number of the ultrasonic modules are allowed to intensively focus on a target region and a smaller number of the ultrasonic modules are allowed to focus on non-target regions.

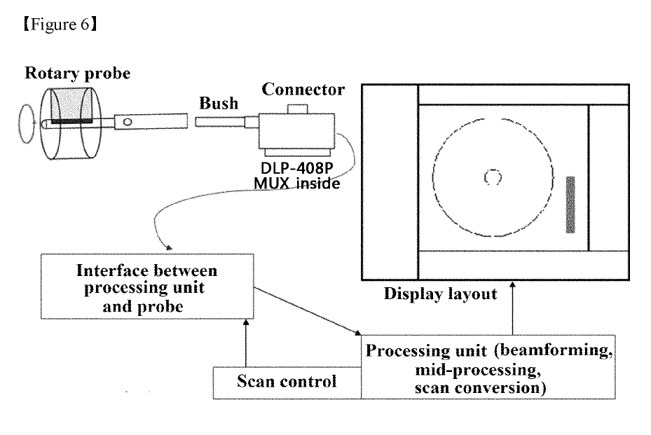

FIG. 5 is a block diagram of an ultrasonic diagnostic system according to one embodiment of the present invention and FIG. 6 is a diagram showing an ultrasonic diagnostic system according to one embodiment of the present invention.

The ultrasonic diagnostic system 500 may include a rotatable element 511 in the form of a linear rod along a rotation axis, a plurality of ultrasonic modules 512 positioned in a row along the rotation axis on the rotatable element, and an image processing unit 520 adapted to create an ultrasound image based on image data acquired during rotation of the ultrasonic modules. The ultrasonic diagnostic system may further include a display unit adapted to provide the ultrasound image.

The rotatable element 511 and the ultrasonic modules 512 constitute a rotary probe 510. The rotary probe 510 of the ultrasonic diagnostic system is the same as the rotary linear probe 100 described with reference to FIGS. 1 to 4 and repeated explanation of the rotary probe 510 is omitted in this description.

The image processing unit 520 creates an ultrasound image based on image data acquired during rotation of the ultrasonic modules.

More specifically, the image processing unit 520 conducts beamforming of ultrasonic signals received by the ultrasonic modules, processes the signals (mid-processing), and can create an image through scan conversion. The creation of an ultrasound image using the received ultrasonic signals is based on any suitable process employed in conventional ultrasound systems.

The display unit 530 displays the ultrasound image to a user.

Although explanatory description is provided with particular features such as specific elements, limited embodiments and drawings, it is to help the more inclusive understanding of the invention and shall not be construed to limit the invention, and it will be appreciated by those skilled in the art in the art that various modifications and changes can be made from the description.

Therefore, the spirit of the present invention shall not be limited to the embodiment described above, and the claims below and their equivalents or any equivalent changes will fall into the scope of the spirit of the invention.

### INDUSTRIAL APPLICABILITY

The use of the rotary linear probe according to the present invention, which includes a rotatable element in the form of a linear rod along a rotation axis and a plurality of ultrasonic modules positioned in a row along the rotation axis on the rotatable element, can provide better diagnostic image information based on image signals with improved 2D/3D circular image quality.

1. A rotary linear probe comprising a rotatable element in the form of a linear rod along a rotation axis and a plurality of ultrasonic modules positioned in a row along the rotation axis on the rotatable element.

2. The rotary linear probe according to claim 1, wherein the plurality of ultrasonic modules are capable of independent focusing.

3. The rotary linear probe according to claim 1, wherein the plurality of ultrasonic modules focus in pairs on different depths corresponding to predetermined planes perpendicular to the rotation axis during rotation to acquire 2D circular cross-sectional image data.

4. The rotary linear probe according to claim 1, wherein the plurality of ultrasonic modules focus on the same depth in a direction perpendicular to the rotation axis during rotating to acquire cylindrical 3D circular image data.

5. The rotary linear probe according to claim 1, wherein each of the ultrasonic modules comprises a transducer adapted to transmit and receive ultrasonic waves and a control unit adapted to adjust the transmission angle of the ultrasonic waves from the transducer.

6. The rotary linear probe according to claim 1, further comprising a rotary motor adapted to rotate the rotatable element.

7. An ultrasonic diagnostic system comprising a rotatable element in the form of a linear rod along a rotation axis, a plurality of ultrasonic modules positioned in a row along the rotation axis on the rotatable element, and an image processing unit adapted to create an ultrasound image based on image data acquired during rotation of the ultrasonic modules.

8. The ultrasonic diagnostic system according to claim 7, further comprising a display unit adapted to display the ultrasound image.
